(19) United States
(12) Patent Application Publication
(10) Pub. No.: US 2023/0256060 A1
(43) Pub. Date: Aug. 17, 2023

(54) Undenatured Type II Collagen as a Supplement for Improved Endurance, Lipid Metabolism, and Oxidative Stress

(71) Applicant: Lonza Greenwood LLC, Greenwood, SC (US)

(72) Inventor: Vijaya Juturu, North Brunswick, NJ (US)

(21) Appl. No.: 18/009,359

(22) PCT Filed: Jun. 9, 2021
(86) PCT No.: PCT/US2021/036591

§ 371 Date: Dec. 9, 2022

Related U.S. Application Data
(60) Provisional application No. 63/038,305, filed on Jun. 12, 2020.

(51) Int. Cl. A61K 38/39; A61P 21/00

(52) U.S. Cl. CPC A61K 38/39; A61P 21/00

(57) ABSTRACT

A supplement for improving one or more of endurance, lipid metabolism, and antioxidant status in a mammal. The supplement includes a type II collagen composition, where the type II collagen composition is present in the supplement in an amount sufficient to improve endurance of a mammal by about 5% or more as compared to a mammal that has not been administered the supplement, improve lipid metabolism by about 5% or more as compared to a mammal that has not been administered the supplement, and/or decrease oxidative stress by about 5% or more as compared to a mammal that has not been administered the supplement.

### BACKGROUND

In recent years, the use of collagen to treat various conditions has become exceedingly popular. Collagen is a protein that can be found in muscles, bones, skin, blood vessels, and in other parts of the body. There are various different types of collagen depending upon its function and form. For instance, Type I collagen, the most abundant collagen, is made of fibers found in tendons, ligaments, organs and skin. Type II collagen, on the other hand, primarily helps build cartilage, a major structural entity that sits on the surfaces of those bones which comprise articulating joints. Type III collagen is a major component of the extracellular matrix that makes up organs and skin. Type III collagen also forms blood vessels and tissue within the heart. Type IV collagen is found primarily in the skin as sheet-like structures in the cutaneous basal lamina. Furthermore, Collagen peptides are portions of one or more of the a strands of any type of collagen formed through enzymatic hydrolysis of collagen. Collagen peptides are often used in beverages and food products, as they are water-soluble and non-gelling.

Collagen has also been found to effectively treat arthritis and other joint pain. For example, U.S. Pat. No. 9,066,926 discloses a method of reducing exercise-induced joint pain in mammals by administering to a mammal Type II collagen. This patent also discloses the mechanism of action through which this ingredient operates: oral tolerance. This putative mechanism entails the stimulation of T regulatory cells (Treg), located in gut associated lymphatic tissue, to specifically recognize antigenic determinants (epitopes) on the native collagen protein. Once induced, the Tregs exit the gut area and migrate to the joint space where they stimulate chondrocytes to lay down new Type II collagen thereby enhancing the structural integrity and flexibility of the articulating joint. One such example, for which clinical data has been published is the knee. The '926 patent is incorporated herein by reference.

Furthermore, there have been studies that demonstrate the importance of physical training on joint strength and flexibility, as well as health benefits, including insulin sensitivity, reduced blood pressure, improved muscle metabolism and improved antioxidant system factors. However, other studies have documented a negative correlation between self-reported physical activity and increased inflammation. Exercise, as well as exposure to sunlight and heat can also result in physiological changes Additionally, exercise has been shown to have a peak endurance that is difficult to increase. Nonetheless, exercise alone has also proven insufficient to improve carbohydrate and lipid metabolism disorders that have become increasingly prevalent.

Therefore, it would be a benefit to provide a supplement that could improve one or more of endurance, lipid metabolism, and antioxidant status. Furthermore, it would be a benefit to provide a supplement that could improve one or more of endurance, lipid metabolism, and antioxidant status, as compared to exercise alone. It would also be a benefit to provide a supplement that could improve one or more of the effects of exercise related to joint health and other health benefits. Furthermore, it would be advantageous to provide compositions and methods which can be used to improve the physiological effects of physical exercise or daily activities, such as to improve quality of life and wellness It would be a further benefit to provide a method of improving one or more of endurance, lipid metabolism, and antioxidant status.

### SUMMARY

The present disclosure is generally directed to a supplement for improving one or more of endurance, lipid metabolism, and antioxidant status in a mammal. The supplement includes a type II collagen composition that includes undenatured collagen, hydrolyzed collagen, or a combination thereof. Furthermore, the type II collagen composition is present in the supplement in an amount sufficient to: increase endurance of a mammal by about 5% or more as compared to a mammal that has not been administered the supplement, improve lipid metabolism by about 5% or more as compared to a mammal that has not been administered the supplement, and/or decrease oxidative stress by about 5% or more as compared to a mammal that has not been administered the supplement.

In one aspect the type II collagen composition is present in the supplement in an amount sufficient to increase a run time to exhaustion in a mammal by about 5% or more as compared to a mammal that has not been administered the supplement. Furthermore, in an aspect, the type II collagen composition is present in the supplement in an amount sufficient to decrease sterol regulatory element binding protein-1c (SREBP-1c) in a mammal by about 5% or more as compared to a mammal that has not been administered the supplement. In yet another aspect, the type II collagen composition is present in the supplement in amount sufficient to decrease malondialdehyde levels in a mammal by about 5% or more as compared to a mammal that has not been administered the supplement.

Moreover, in an aspect, the type II collagen composition further comprises one or more different types of collagen in addition to the type II collagen. Additionally or alternatively, wherein the one or more different types of collagen include native type II collagen, collagen peptides, or a mixture thereof. Furthermore, in an aspect, the collagen is undenatured type II collagen, and is derived from chicken sternum cartilage. In one aspect, the undenatured type II collagen comprises about 1% to about 95% of the type II collagen composition. In yet another aspect, the undenatured type II collagen has a total oxygen radical absorbance capacity of about 200 μmol TE/g or greater, as measured according to ORAC 6.0.

Additionally or alternatively, the supplement is in the form of, or is incorporated into, a food or beverage.

Furthermore, the present disclosure is also generally directed to a method of improving one or more of endurance, lipid metabolism, and antioxidant status in a mammal. The method includes administering a supplement to the mammal that includes a type II collagen composition, where the type II collagen composition includes undenatured collagen, hydrolyzed collagen, or a combination thereof. Furthermore, the supplement is administered in an amount sufficient to increase endurance of a mammal by about 5% or more as compared to a mammal that has not been administered the supplement, improve lipid metabolism by about 5% or more as compared to a mammal that has not been administered the supplement, and/or decrease oxidative stress by about 5% or more as compared to a mammal that has not been administered the supplement.

In one aspect, both the mammal administered the supplement, and a mammal that has not been administered the supplement, have previously undergone physical activity, are currently undergoing physical activity, or have previously undergone physical activity and are currently undergoing physical activity. In another aspect, the method further includes subjecting both the mammal administered the supplement, and a mammal that has not been administered the supplement to a time of physical activity, wherein the increase in endurance, the improved lipid metabolism, and/or the decrease in oxidative stress is measured after the time of physical activity. Moreover, in an aspect, the time of physical activity is at least one week, and wherein the increase in endurance, the improved lipid metabolism, and/or the decrease in oxidative stress is measured at the end of the at least one week.

In a further aspect, the type II collagen composition is present in the supplement in an amount sufficient to increase a run time to exhaustion in a mammal by about 5% or more as compared to a mammal that has not been administered the supplement. Additionally or alternatively, the type II collagen composition is present in the supplement in an amount sufficient to decrease sterol regulatory element binding protein-1c (SREBP-1c) in a mammal by about 5% or more as compared to a mammal that has not been administered the supplement. Furthermore, in an aspect, the type II collagen composition is present in the supplement in amount sufficient to decrease malondialdehyde levels in a mammal by about 5% or more as compared to a mammal that has not been administered the supplement.

Additionally or alternatively, in an aspect, the type II collagen composition is administered to the mammal daily. Moreover, in an aspect, the type II collagen composition is administered to a mammal while the mammal is undergoing physical activity. In yet another aspect, the type II collagen composition is administered to the mammal as a food or beverage.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1A shows graphs determined using the Western blot technique for several factors according to the present disclosure;

FIG. 1B shows graphs determined using the Western blot technique for several factors according to the present disclosure;

FIG. 1C shows graphs determined using the Western blot technique for several factors according to the present disclosure;

Repeat use of reference characters in the present specification and drawing is intended to represent same or analogous features or elements of the invention.

### Definitions

As used herein, the terms “about,” “approximately,” or “generally,” when used to modify a value, indicates that the value can be raised or lowered by 10%, in one aspect, such as 8%, such as 5% in one aspect, and remain within the disclosed aspect.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation canine, equine, feline, bovine, ovine, human, or porcine mammals.

As used herein, “healthy” refers to the absence of illness or injury.

The term “physical activity” means activity that lasts about 10 minutes or more, such as about 25 minutes or more, such as about 30 minutes or more, such as at least about 45 minutes or more, and where the heart rate of the mammal reaches about 30% to about 85% of its maximum heart rate, such as about 40% to about 80%, such as about 50% to about 75% of the maximum heart rate of the mammal.

The term “intensive physical activity” means activity that lasts about 20 minutes or more, such as about 25 minutes or more, such as about 30 minutes or more, such as at least about 45 minutes or more, and where the heart rate of the mammal reaches about 50% to about 99% of its maximum heart rate, such as about 55% to about 95%, such as about 60% to about 90%, such as about 705 to about 85% of the maximum heart rate of the mammal.

The term “endurance” means an ability of a mammal to exert itself and remain active for a long period of time. Thus, in one aspect of the present disclosure, an improvement in endurance includes an increase in the amount of time of the physical activity, or the level of exertion during the physical activity over the same amount of time, or a combination thereof.

The term “sports nutrition” includes an improvement in recovery, explosive strength, rate of response, joint mobility, range of motion index, flexibility, and/or stretching, or a combination thereof.

Unless otherwise noted, “collagen” as used herein refers to all forms of collagen, either with or without denaturation, without or without salts or stabilizing agents, and fibrillar and non-fibrillar types of collagen not limited to fibril associated collagens with interrupted triple helices (FACIT, Type IX, XII, XIV, XIX, XXI), including short chain collagen (generally Types VII and X), basement membrane (Type IV), Multiplexin (multiple triple helix domains with interruptions (Type XV, XVIII), and other types of collagen (Types VI, VII).

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a supplement for improving one or more of endurance, lipid metabolism, and antioxidant status. Particularly, the present disclosure has found that a type II collagen supplement unexpectedly improves one or more of endurance, lipid metabolism, and antioxidant status in a mammal, as compared to the same mammal that was not administered the supplement. In particular, an undenatured Type-II collagen supplement was found to be effective for the purposes of this disclosure. Furthermore, the present disclosure has also found that such benefits may be displayed, or further improved if the mammal has undergone physical or intensive physical activity, is currently undergoing physical activity or intensive physical activity, or has both undergone and is undergoing physical or intensive physical activity, even as compared to a mammal that has similarly undergone and/or is undergoing physical or intensive physical activity.

For instance, the present disclosure has found that a mammal administered a type II collagen supplement according to the present disclosure may exhibit an improvement in run time to exhaustion as compared to the same mammal that has not been administered the undenatured type II collagen supplement, of about 5% or greater, such as about 10% or greater, such as about 12.5% or greater, such as about 15% or greater, such as about 17.5% or greater.

In one aspect, the mammal may be trained, meaning that the mammal administered the supplement, the mammal that was not administered the supplement, or both the mammal administered the supplement and the mammal that was not administered the supplement, have undergone physical activity or intensive physical activity. In one aspect, the physical activity and/or intensive physical activity make take place for a period of time of about 1 day or more, such as about 5 days or more, such as about 1 week or more, such as about 2 weeks or more, such as about 3 weeks or more, such as about 4 weeks or more, such as about 6 weeks or more, such as about 8 weeks or more. In one aspect, the physical activity or intensive physical activity may be conducted in conjunction with supplementation (e.g. type II collagen is administered to the mammal while the mammal is undergoing training), where the increase in endurance is measured at the end of one or more of the above time periods. Alternatively, in an aspect, the mammal is already trained, and is administered the supplement after training, and the increase in endurance is measured after supplementation according to one or more of the above time periods. In yet a further aspect, the mammal may be trained prior to supplementation, and may also undergo further training in conjunction with the type II collagen supplementation.

Nonetheless, the present disclosure has further found that supplementation with a type II collagen supplement according to the present disclosure, either alone or in conjunction with physical or intensive physical activity as discussed above, also surprisingly resulted in a decrease in lipid metabolism markers, including sterol regulatory element binding protein-1c (SREBP-1c), ATP citrate lyase (ACLY), liver X receptors (LXRs), and fatty acid synthase (FAS). For instance, SREPB-1c regulates genes required for glucose metabolism, fatty acid production, and lipid production. Thus, reduced levels of SREBP-1c may indicated reduced insulin resistance. Similarly, ACLY is an enzyme used during fatty acid biosynthesis. Particularly, by converting citrate to acetyl-CoA, ACLY links carbohydrate metabolism with fatty acid biosynthesis. Furthermore, LXRs are a member of the nuclear receptor family of transcription factors, and are closely related to nuclear receptors such as peroxisome proliferator-activated receptors (PPARs), farnesoid X receptor (FXR), and retinoid X receptor (RXR). These receptors, for instance, the liver X receptors, are important regulators of cholesterol, fatty acid, and glucose homeostasis. Furthermore, fatty acid synthase (FAS) is an enzyme encoded by the FASN gene. Fatty acid synthase is a multi-enzyme protein that catalyzes fatty acid synthesis. Thus, it is clear that improved modulation of one or more these markers may evidence improved carbohydrate and lipid metabolism.

For instance, the present disclosure has found that a mammal administered a type II collagen supplement according to the present disclosure may exhibit a decrease in SREPB-1 as compared to the same mammal that has not been administered the type II collagen supplement of about 5% or more, such as about 7.5% or more, such as about 10% or more, such as about 12.5% or more, such as about 15% or more, such as about 17.5% or more, such as about 20% or more, such as about 22.5% or more, such as about 25% or more.

Moreover, in one aspect, as discussed above, the mammal administered a type II collagen supplement according to the present disclosure may exhibit a decrease in SREPB-1 as compared to the same mammal that has not been administered the type II collagen supplement, even when both animals are trained, are currently undergoing physical or intensive physical activity, or are both trained and undergoing physical or intensive physical activity. For instance, in one aspect, a mammal administered a type II collagen supplement according to the present disclosure may exhibit a decrease in SREPB-1 as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 5% or more, such as about 10% or more, such as about 12.5% or more, such as about 15% or more, such as about 17.5% or more, such as about 20% or more, such as about 22.5% or more.

Similarly, in one aspect, a mammal administered a type II collagen supplement according to the present disclosure may exhibit a decrease in ACLY as compared to the same mammal that has not been administered the type II collagen supplement of about 5% or more, such as about 10% or more, such as about 12.5% or more, such as about 15% or more, such as about 17.5% or more, such as about 20% or more, such as about 22.5% or more, such as about 25% or more.

In one aspect, a mammal administered a type II collagen supplement according to the present disclosure may exhibit a decrease in LXRs as compared to the same mammal that has not been administered the type II collagen supplement of about 5% or more, such as about 10% or more, such as about 12.5% or more, such as about 15% or more, such as about 17.5% or more, such as about 20% or more, such as about 22.5% or more.

Moreover, in one aspect, as discussed above, the mammal administered a type II collagen supplement according to the present disclosure may exhibit a decrease in LXRs as compared to the same mammal that has not been administered the type II collagen supplement, even when both animals are trained, are currently undergoing physical or intensive physical activity, or are both trained and undergoing physical or intensive physical activity. For instance, in one aspect, a mammal administered a type II collagen supplement according to the present disclosure may exhibit a decrease in LXRs as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 2.5% or more, such as about 5% or more, such as about 7.5% or more, such as about 10% or more, such as about 12.5% or more.

In a further aspect, a mammal administered a type II collagen supplement according to the present disclosure may exhibit a decrease in FAS as compared to the same mammal that has not been administered the type II collagen supplement of about 5% or more, such as about 10% or more, such as about 12.5% or more, such as about 15% or more, such as about 17.5% or more, such as about 20% or more, such as about 22.5% or more, such as about 25% or more.

Additionally, the present disclosure has found that supplementation with a type II collagen supplement according to the present disclosure, either alone or in combination with physical activity or intensive physical activity, also resulted in a decrease in inflammatory markers such as interleukin 1-beta (Il-1β) and tumor necrosis factor alpha (TNF-α). For instance, in one aspect, a mammal administered a type II collagen supplement according to the present disclosure may exhibit a decrease in Il-1β as compared to the same mammal that has not been administered the undenatured type II collagen supplement of about 5% or more, such as about 10% or more, such as about 12.5% or more, such as about 15% or more, such as about 17.5% or more, such as about 20% or more, such as about 22.5% or more. Furthermore, in one aspect, the supplement of the present disclosure may also decrease inflammatory cytokines, or other negative inflammatory markers associated with sport nutrition and exercise physiology.

Moreover, in one aspect, as discussed above, the mammal administered a type II collagen supplement according to the present disclosure may exhibit a decrease in Il-1β as compared to the same mammal that has not been administered the type II collagen supplement, even when both animals are trained, are currently undergoing physical or intensive physical activity, or are both trained and undergoing physical or intensive physical activity. For instance, in one aspect, a mammal administered a type II collagen supplement according to the present disclosure may exhibit a decrease in Il-1β as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 2.5% or more, such as about 5% or more, such as about 7.5% or more, such as about 10% or more.

Similarly, in an aspect, a mammal administered a type II collagen supplement according to the present disclosure may exhibit a decrease in TNF-α as compared to the same mammal that has not been administered the type II collagen supplement of about 5% or more, such as about 10% or more, such as about 12.5% or more, such as about 15% or more, such as about 17.5% or more, such as about 20% or more, such as about 22.5% or more.

Moreover, in one aspect, as discussed above, the mammal administered a type II collagen supplement according to the present disclosure may exhibit a decrease in TNF-α as compared to the same mammal that has not been administered the type II collagen supplement, even when both animals are trained, are currently undergoing physical or intensive physical activity, or are both trained and undergoing physical or intensive physical activity. For instance, in one aspect, a mammal administered a type II collagen supplement according to the present disclosure may exhibit a decrease in TNF-α as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 2.5% or more, such as about 5% or more, such as about 7.5% or more, such as about 10% or more.

Additionally, the present disclosure has found that supplementation with a type II collagen supplement according to the present disclosure, either alone or in combination with physical activity or intensive physical activity, also resulted in improvement in muscle health and growth. For instance, muscle atrophy F-box-1 (MAFbx) and muscle RING-finger protein-1 (MuRF-1) participate in skeletal muscle atrophy, and reduction in MAFbx and MuRF-1 show decreases in muscle atrophy. Similarly, myoblast determination protein-1 (MyoD) regulates muscle differentiation, and is shown to decrease as mammals age. Furthermore, myostatin is a protein produced and released by myocytes that inhibits muscle growth. Additionally, creatine kinase indicates damage or disease to skeletal muscle.

Thus, in one aspect, a mammal administered a type II collagen supplement according to the present disclosure may exhibit a decrease in MAFbx as compared to the same mammal that has not been administered the undenatured type II collagen supplement of about 5% or more, such as about 10% or more, such as about 12.5% or more, such as about 15% or more, such as about 17.5% or more, such as about 20% or more, such as about 22.5% or more

Moreover, in one aspect, as discussed above, the mammal administered a type II collagen supplement according to the present disclosure may exhibit a decrease in MAFbx as compared to the same mammal that has not been administered the type II collagen supplement, even when both animals are trained, are currently undergoing physical or intensive physical activity, or are both trained and undergoing physical or intensive physical activity. For instance, in one aspect, a mammal administered a type II collagen supplement according to the present disclosure may exhibit a decrease in MAFbx as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 2.5% or more, such as about 5% or more, such as about 7.5% or more, such as about 10% or more.

Furthermore, in one aspect, a mammal administered a type II collagen supplement according to the present disclosure may exhibit a decrease in MuRF-1 as compared to the same mammal that has not been administered the type II collagen supplement of about 5% or more, such as about 10% or more, such as about 15% or more, such as about 20% or more, such as about 25% or more, such as about 30% or more, such as about 35% or more, such as about 40% or more, such as about 45% or more, such as about 47.5% or more, such as about 50% or more, such as about 52.5% or more.

Moreover, in one aspect, as discussed above, the mammal administered a type II collagen supplement according to the present disclosure may exhibit a decrease in MuRF-1 as compared to the same mammal that has not been administered the type II collagen supplement, even when both animals are trained, are currently undergoing physical or intensive physical activity, or are both trained and undergoing physical or intensive physical activity. For instance, in one aspect, a mammal administered a type II collagen supplement according to the present disclosure may exhibit a decrease in MuRF-1 as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 5% or more, such as about 10% or more, such as about 15% or more, such as about 17.5% or more, such as about 20% or more, such as bout 22.5% or more.

In yet a further aspect, a mammal administered a type II collagen supplement according to the present disclosure may exhibit an increase in MyoD as compared to the same mammal that has not been administered the type II collagen supplement of about 50% or more, such as about 55% or more, such as about 60% or more, such as about 65% or more, such as about 70% or more, such as about 75% or more, such as about 80% or more, such as about 85% or more, such as about 90% or more, such as about 95% or more, such as about 100% or more, such as about 105% or more, up to about 150%.

Similarly, in one aspect, a mammal administered a type II collagen supplement according to the present disclosure may exhibit a decrease in myostatin as compared to the same mammal that has not been administered the type II collagen supplement of about 5% or more, such as about 10% or more, such as about 12.5% or more, such as about 15% or more, such as about 17.5% or more, such as about 20% or more, such as about 22.5% or more.

Further, in one aspect, as discussed above, the mammal administered a type II collagen supplement according to the present disclosure may exhibit a decrease in myostatin as compared to the same mammal that has not been administered the type II collagen supplement, even when both animals are trained, are currently undergoing physical or intensive physical activity, or are both trained and undergoing physical or intensive physical activity. For instance, in one aspect, a mammal administered a type II collagen supplement according to the present disclosure may exhibit a decrease in myostatin as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 2.5% or more, such as about 5% or more, such as about 7.5% or more, such as about 10% or more, such as about 12.5% or more.

Additionally, in one aspect, a mammal administered a type II collagen supplement according to the present disclosure may exhibit a decrease in creatine kinase as compared to the same mammal that has not been administered the type II collagen supplement, where both animals are trained, are currently undergoing physical or intensive physical activity, or are both trained and undergoing physical or intensive physical activity. For instance, in one aspect, a mammal administered a type II collagen supplement according to the present disclosure may exhibit a decrease in creatine kinase as compared to the same mammal that has not been administered the type II collagen supplement, where both mammals are trained, undergoing physical or intensive physical activity along with the supplementation as described above, or both trained and undergoing physical or intensive physical activity along with supplementation as described above, of about 2.5% or more, such as about 5% or more, such as about 7.5% or more, such as about 10% or more.

The present disclosure has found that supplementation with a type II collagen supplement according to the present disclosure, either alone or in combination with physical activity or intensive physical activity, also resulted in improvement in oxidative stress. For instance, in one aspect, a mammal administered a type II collagen supplement according to the present disclosure may exhibit a decrease in malondialdehyde levels as compared to the same mammal that has not been administered the type II collagen supplement of about 5% or more, such as about 7.5% or more, such as about 10% or more, such as about 12.5% or more, such as about 14% or more. Additionally or alternatively, in one aspect, a mammal administered a type II collagen supplement according to the present disclosure may exhibit an increase in superoxide dismutase enzyme (SOD) as compared to the same mammal that has not been administered the type II collagen supplement of about 1% or more, such as about 2% or more, such as about 2.5% or more, such as about 3% or more, such as about 3.3% or more.

Nonetheless, in one aspect, the type II collagen according to the present disclosure is incorporated into the supplement as a collagen composition. The collagen composition may include one or more of any collagen as defined above, and/or, in one aspect, may include one or more of Type I collagen, Type II collagen, Type III collagen, Type IV collagen, or collagen peptides, or a mixture thereof. In one aspect, the collagen composition contains Type II collagen alone or in combination with one or more of Type I collagen, Type III collagen, Type IV collagen, or collagen peptides. In one aspect, the collagen composition may include a mixture of type II collagen (sometimes referred to as native type II collagen) and undenatured type II collagen. Additionally or alternatively, the collagen composition may include a mixture of native type II collagen and undenatured type II collagen, in addition to a further collagen, such as Type I, Type III, Type IV, or collagen peptides. Furthermore, in one aspect, the collagen composition includes whole collagen protein, biologically active peptide fragments of collagen, or a combination thereof.

In one aspect, one or more types of collagen in the collagen composition may be heat sterilized, such as by autoclaving, and/or may also include salts, such as alkalizing or acid, and/or organic or inorganic salts. Thus, in one aspect, a portion of the collagen may be at least partially hydrolyzed. In one aspect, the collagen is hydrolyzed by any process or compound, including by an acid base agent, an enzyme, heat or other temperature extreme, a chemical, UV, a salt, or combinations thereof. In one aspect, the hydrolyzed collagen is undenatured collagen that has been at least partially hydrolyzed by any method, and in one aspect, may include undenatured collagen that has any portion that has been denatured. However, as discussed, in one aspect, at least a portion of the collagen composition is undenatured.

As indicated above, in one aspect, the supplement contains a collagen composition, particularly a Type II collagen composition such as an undenatured Type II collagen composition, an at least partially hydrolyzed Type II collagen composition, or a combination thereof. Type II collagen for use in the present disclosure can be obtained from any suitable source. For instance, the collagen can be derived from a variety of mammalian sources, avian sources, or can be obtained from various fish species or a combination thereof. For instance, the collagen can be obtained from salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II® brand from Lonza Consumer Health. UC-II® brand is a natural ingredient that contains a glycosylated, undenatured Type II collagen. The collagen composition can also comprise a hydrolyzed collagen. The collagen composition can also comprise a pure protein or active peptide fragments. In one embodiment, the collagen composition can be free of any bone or bone material. In other embodiments, the collagen composition can be free of any transforming growth factors (TGFs), bone morphogenetic proteins (BMPs), or both. In still another embodiment, the collagen composition comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the type II collagen in the tissue, such as low-temperature processing, and formed into doses containing therapeutically effective levels of undenatured type II collagen, said levels being generally in the amount of at least about 0.01 gram and preferably from about 0.02 to about 0.5 grams of animal tissue in a dose. Being a natural product some variation from sample to sample is to be expected. These variations can be minimized by blending after comminution. The blending can be aided by analytical techniques which allow the measurement of the amount of undenatured type II collagen and other constituents.

Nonetheless, the present disclosure has found that by carefully forming the particles and sterilizing the type II collagen as discussed above, the undenatured type II collagen may be resistant to gastric acid and digestive enzymes in the stomach. Due to this sterilization process, the undenatured type II collagen also retains its 3-dimensional shape, preserving the bioactive epitope regions. Without wishing to be bound by theory, it is believed that the epitope regions contain the ability to induce oral tolerance as discussed above. Particularly epitope regions allow undenatured collagen to bind to the Peyer's Patches, which have the ability to induce oral tolerance processes.

In one aspect, the collagen composition is present in the supplement in an amount from about 1 milligram to about 5000 milligrams. For instance, the collagen composition can be present in the supplement in an amount greater than about 5 milligrams, such as in an amount greater than about 10 milligrams, such as in an amount greater than about 15 milligrams, such as in an amount greater than about 20 milligrams, such as in an amount greater than about 25 milligrams, such as in an amount greater than about 30 milligrams. The total amount of collagen composition present in the supplement is generally less than about 1000 milligrams, such as less than about 900 milligrams, such as less than about 800 milligrams, such as less than about 700 milligrams, such as less than about 600 milligrams, such as less than about 500 milligrams, such as less than about 400 milligrams, such as less than about 300 milligrams, such as less than about 200 milligrams, such as less than about 100 milligrams, such as less than about 80 milligrams, such as less than about 70 milligrams, such as less than about 60 milligrams, or any ranges or values therebetween. Furthermore, it should be understood that, in one aspect, the collagen composition may be a type II collagen composition, where substantially all of the collagen in the collagen composition is type II collagen.

In one aspect, undenatured type II collagen may form all, or substantially all, of the total type II collagen in the collagen composition, and therefore, may be present in the supplement in the above discussed amounts. However, in one aspect, undenatured type II collagen may account for about 1% to about 95% of the total type II collagen and/or collagen composition, such as about 2.5% to about 75%, such as about 5% to about 50%, such as about 10% to about 40% of the total type II collagen or total collagen composition, or any ranges or values therebetween. Therefore, in one aspect, undenatured type II collagen may be present in the composition in an amount of 0.1 mg to about 100 mg, such as about 0.5 mg to about 75 mg, such as about 0.75 mg to about 50 mg, such as about 1 mg to about 30 mg, or any ranges or values therebetween.

Furthermore, in one aspect, the collagen composition may further include a preservative salt, such as potassium chloride. Thus, in one aspect, the total amounts of collagen composition discussed above may include type II collagen and/or undenatured type II collagen, alone or in combination with a further collagen, a preservative salt, or combinations thereof. In such as aspect, the total type II collagen, including native and undenatured type II collagen, may account for about 1% to about 99% of the collagen composition, such as about 2.5% to about 90%, such as about 5% to about 80%, such as about 7.5% to about 70%, such as about 10% to about 60%, such as about 15% to about 50%, such as about 20% to about 35%, or any ranges or values therebetween. Thus, in one aspect, the total amount of type II collagen, including native and undenatured type II collagen in the collagen composition may be from about 1 mg to about 1000 mg, such as about 2.5 mg to about 500 mg, such as about 5 mg to about 250 mg, such as about 7.5 mg to about 100 mg, such as about 10 mg to about 40 mg, or any ranges or values therebetween. Of course, in one aspect, no preservative salt is used.

Furthermore, in one aspect, when the type II collagen includes undenatured type II collagen, the undenatured type II collagen may have a large oxygen radical absorbance capacity (ORAC), as measured according to ORAC 6.0. Particularly, ORAC tests measure antioxidant scavenging activity against oxygen radicals that are known to be involved in the pathogenesis of aging and common disease, and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as μmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2,2′ azobis(2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosyndnonimine hydrochloride. The ORAC assay against superoxide measures the antioxidant capacity of the sample to protect hydroethidine from damage by a superoxide which is generated from xanthine oxidase. The ORAC assay against singlet oxygen measures the antioxidant capacity of the sample to protect hydroethidine from damage by single oxygen which is generated from a reaction between lithium molybdate and hydrogen peroxide. Finally, the ORAC assay against hypochlorite measures the antioxidant capacity of the sample to protect the fluorescent protein fluorescein from damage by the hypochlorite radical which is generated from sodium hypochlorite.

Thus, in one aspect, a collagen composition having an undenatured type II collagen according to the present disclosure may have a total ORAC of about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 600 μmol TE/g or greater, such as about 700 μmol TE/g or greater, such as about 750 μmol TE/g or greater, such as about 800 μmol TE/g or greater, such as about 825 μmol TE/g or greater, up to about 1000 μmol TE/g, or any ranges or values therebetween.

Furthermore, in one aspect, a collagen composition having an undenatured type II collagen according to the present disclosure may have a ORAC against peroxyl radicals of about 1 μmol TE/g or greater, such as about 2.5 μmol TE/g or greater, such as about 5 μmol TE/g or greater, such as about 7.5 μmol TE/g or greater, such as about 10 μmol TE/g or greater, such as up to about 10.5 μmol TE/g or greater, up to about 50 μmol TE/g, or any ranges or values therebetween.

Similarly, in one aspect, a collagen composition having an undenatured type II collagen according to the present disclosure may have a ORAC against hydroxyl radicals of about 10 μmol TE/g or greater, such as about 15 μmol TE/g or greater, such as about 20 TE/g or greater, such as about 25 μmol TE/g or greater, such as about 27.5 μmol TE/g or greater, such as about 30 mol TE/g or greater, up to about 40 mol TE/g, or any ranges or values therebetween.

Additionally or alternatively, in one aspect, a collagen composition having an undenatured type II collagen according to the present disclosure may have a ORAC against peroxynitrite of about 0.5 μmol TE/g or greater, such as about 1 μmol TE/g or greater, such as about 1.5 μmol TE/g or greater, such as about 2 mol TE/g or greater, such as about 2.25 TE/g or greater, up to about 5 μmol TE/g, or any ranges or values therebetween.

In one aspect, a collagen composition having an undenatured type II collagen according to the present disclosure may have a ORAC against singlet oxygen of about 500 TE/g or greater, such as about 550 μmol TE/g or greater, such as about 600 mol TE/g or greater, such as about 650 μmol TE/g or greater, such as about 700 mol TE/g or greater, such as about 725 μmol TE/g or greater, up to about 1000 mol TE/g, or any ranges or values therebetween.

Furthermore, in one aspect, a collagen composition having an undenatured type II collagen according to the present disclosure may have a ORAC against hypochlorite of about 25 μmol TE/g or greater, such as about 30 mol TE/g or greater, such as about 35 TE/g or greater, such as about 40 mol TE/g or greater, such as about 45 μmol TE/g or greater, such as up to about 50 μmol TE/g or greater, up to about 75 μmol TE/g, or any ranges or values therebetween.

Furthermore, in one aspect, when the type II collagen includes undenatured type II collagen, the undenatured type II collagen may have a molecular weight of about 10,000 Daltons or more, such as about 15,000 Daltons or more, such as about 20,000 Daltons or more, such as about 25,000 Daltons or more, such as about 30,000 Daltons or more, such as about 35,000 Daltons or more, such as about 40,000 Daltons or more, such as about 45,000 Daltons or more, such as about 50,000 Daltons or more, such as about 55,000 Daltons or more, such as about 60,000 Daltons or more, such as about 65,000 Daltons or more, such as about 70,000 Daltons or more, such as about 75,000 Daltons or more, such as about 80,000 Daltons or more, such as about 85,000 Daltons or more, such as about 90,000 Daltons or more such as about 95,000 Daltons or more, such as about 100,000 Daltons or more, up to about 350,000 Daltons or less, or any ranges or values therebetween.

While various aspects and benefits have been discussed, in one aspect, the collagen composition is incorporated into a suitable delivery form prior to incorporation into a dosage form as discussed below. In one aspect, the composition of the present disclosure may be included as an oil-in-water emulsion as a delivery form. Particularly, in one aspect, such an arrangement may allow one or more oil-soluble and/or one or more water-soluble active ingredients to be contained in the same delivery form. Alternatively, only oil-soluble components may be used (e.g. the Type II collagen), and the emulsion may be used to incorporate the composition into a water-based application.

Nonetheless, the oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible. In some embodiments, the gum arabic may be comprised of a 100% modified gum arabic, such as Ticamulsion® A-2010 gum arabic powder. In certain embodiments, the gum arabic may be a mixture or blend of gum arabic and modified gum arabic. For example, in certain embodiments, the gum arabic may comprise Ticamulsion® 3020.

In certain aspects, the oil-in-water emulsion contains from about 10% to about 30% by weight of gum arabic. In some embodiments, the oil-in-water emulsion contains from about 15% to about 25% by weight of gum arabic. In some embodiments, the oil-in-water emulsion contains less than about 20% by weight of gum arabic, such as less than 15%, such as less than 10%, such at less than 5%.

The oil-in-water emulsion may also contain water. In certain aspects, the oil-in-water emulsion contains deionized water. Still, in certain aspects, the oil-in-water emulsion may contain any water suitable for human ingestion and incorporation into dietary supplements designed for human ingestion.

The amount of water incorporated into the oil-in-water emulsion can vary depending on the desired hygroscopic and water-soluble ingredients that are incorporated into the oil-in-water emulsion. In certain aspects, the oil-in-water emulsion may contain from about 5% to 35% by weight of water. In some embodiments, the oil-in-water emulsion may contain from about 10% to about 30% by weight of water. In some embodiments, the oil-in-water emulsion may contain from about 15% to about 20% by weight of water. In some embodiments, the oil-in-water emulsion may contain less than about 20% by weight of water, such as less than about 15% by weight of water, such as less than about 10% by weight of water.

In some aspects, the oil-in-water emulsion may contain one or more stabilizers or suspension promoting agents. For example, in certain aspects, the oil-in-water emulsion may contain one or more gum, such as gellan gum or xanthum gum. If included, the gellan gum or xanthum gum may be present in an amount of less than about 3.5% by weight of the oil-in-water emulsion, such as less than about 2.5% by weight, such as less than about 1.5% by weight, such as less than about 1.0% by weight, such as less than about 1.0% by weight.

In other aspects, the oil-in-water emulsion may contain one or more stabilizers such as silica. If included, silica may be present in an amount of less than about 2% by weight, such as less than about 1.5% by weight, such as less than about 1% by weight, such as less than about 0.5% by weight.

Furthermore, in one aspect, the oil-in-water emulsion may also contain one or more fat-soluble ingredients or nutrients. In certain aspects, the one or more fat-soluble ingredients or nutrients may be incorporated into the oil phase of the oil-in-water phase emulsion. Suitable fat-soluble ingredients include, but are not limited to retinol, vitamin E sourced from mixed tocopherols, beta carotene, ubiquinone, lecithin, sunflower lecithin, vitamin D, cannabinoids, hemp extracts, vitamin K, phosphatidyl choline, and combinations thereof.

In certain aspects, at least one or more fat-soluble ingredients may be incorporated in the oil-in-water emulsion in an amount of from about 0% by weight to about 50% by weight. For example, in some aspects, the oil-in-water emulsion contains less than about 50% by weight of one or more fat-soluble ingredients, such as less than about 40% by weight, such as less than about 30% by weight, such as less than about 20% by weight, such as less than about 10% by weight, such a less than about 5% by weight.

Moreover, in one aspect, the oil-in water emulsion may contain one or more additional antioxidants, in one or more of the water soluble phase, or the oil/fat soluble phase.

In some aspects, the oil-in-water emulsion disclosed herein may be used any suitable dosage form, such as tablets, gummy chewables, edible films, lozenges, liquid suspensions, syrups, lipid micelles, spray-dried dispersions, nanoparticles, and the like, which may also be incorporated into a further supplement.

Alternatively, the oil-in-water emulsion may be contained in a nutritional product, such as a food product or in a beverage. For example, in certain aspects, the oil-in-water emulsion may be incorporated into a liquid nutritional product, such as a nutritional supplement or infant formula, to be consumed by a mammal. Furthermore, the oil-in-water emulsions provided herein may be added to any liquid nutritional product designed to provide nutritional supplementation to a mammal.

The food or beverage composition may comprise any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, or the like.

Nonetheless, in one aspect, the supplement according to the present disclosure may be administered to the mammal including by oral, enteral or by-inhalation administration of whole collagen protein or biologically active peptide fragments of collagen. For instance, in one aspect, it is believed that the whole collagen protein or biologically active peptide fragments of collagen enhances the content of trans-L-hydroxyproline based on the total weight of amino acids contained in collagen type II, and enables efficient production of trans-L-hydroxyproline to enhance the efficacy.

The supplement composition of the present disclosure may further comprise one or more excipients as further additives in the composition. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, fats, oils and/or waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the supplement composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the supplement composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the supplement composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Consumer Health Inc, having offices in Morristown, N.J. USA.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the supplement composition may comprise silica. For instance, the oxide particles may comprise precipitated silica particles. The silica particles may have a particle size (d50, laser defraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Furthermore, in one aspect, the supplement may be formulated into a food and/or beverage for sports or daily nutritional purposes. In such an aspect, the supplement may further include at least one vitamin, such as at least one of vitamin B, vitamin C, and vitamin E. Vitamins may be contained in the supplement in an amount of from about 50 μg/g of supplement to about 5000 μg/g, such as about 100 μg/g to about 4500, such as about 250 μg/g to about 4000 μg/g, such as about 400 μg/g to about 3500 μg/g, or any ranges or values therebetween. The above ranges may be for any one vitamin alone or a total amount of all vitamins. In one aspect, vitamin E is present in supplement in an amount of about 100 μg/g to about 1000 μg/g, such as about 250 μg/g to about 750 μg/g, such as about 400 μg/g to about 600 μg/g, or any ranges or values therebetween. In another aspect, vitamin C is present in supplement in an amount of about 1000 μg/g to about 5000 μg/g, such as about 2000 μg/g to about 4000 μg/g, such as about 3000 μg/g to about 3750 μg/g, or any ranges or values therebetween.

Furthermore, in an aspect, the supplement contains at least one mineral, such as at least one of potassium magnesium, zinc, or calcium. Minerals may be contained in the supplement in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the supplement contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the supplement contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the supplement contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

Additionally, the supplement may further include at least one additive that enhances sports performance or that contributes to reducing oxidative stress, such as an additionally antioxidant in combination with the collagen composition. For instance, in one aspect, an additive may be one or more of curcumin, spirulina, astaxanthin, or other carotenoids. Furthermore, in one aspect, the present disclosure may include one or more microalgae with a high superoxide dismutase (SOD) and/or ORAC level. Particularly, such microalgae may further help to reduce oxidative stress, and may contribute further anti-inflammatory properties and protection against infections, including improvement in immune health.

Moreover, in one aspect, an additive may include one or more prebiotics and/or probiotics. For instance, in one aspect, anti-fatigue probiotic bacteria, or prebiotic, for improving exercise performance, particularly the fatigue caused by exercise, may be included in the supplement to further improved one or more of endurance. Particularly, probiotic bacteria may further increase muscle mass and endurance, decreases blood lactate, ammonia, and creatine kinase concentration, and change body composition including metabolic health markers, as compared to the collagen composition alone.

Similarly, in one aspect, the collagen composition may be combined with a protein supplement, a creatine supplement, whey protein concentrate, whey protein isolate, or a combination thereof. Furthermore, in an aspect, the supplement can also include a cannabigerol, such as cannabidiol.

Furthermore, in an aspect, the supplement of the present disclosure can also include a mineral and branched-chain amino acid chelate, alone or in addition to a branched chain amino acid. Particularly, it has been found that a mineral and branched-chain amino acid chelate can increase absorption of the branched-amino acid in a mammal. Such an uptake may increase or maintain muscle mass in trained mammals or in individuals having diseases such as ALS, muscular dystrophy, sarcopenia associated with aging, or muscle atrophy associated with spinal cord injury. Furthermore, in an aspect, a mineral and branched-chain amino acid chelate as well as to decrease mental fatigue. Nonetheless, in one aspect, the branched chain amino acid may be one or more of leucine, isoleucine, and valine, and chelating a mineral with the one or more branched-chain amino acids. In one aspect, the mineral may be calcium, magnesium, zinc, or a mixture thereof. However, it should be understood that the mineral may also be a mineral source, such as a carbonate of the one or more minerals.

Nonetheless, in one aspect, for instance, the supplement of the present disclosure may also be formulated to improve joint health, muscle health, cartilage heath, ligament heath, tendon health, bone health, or combinations thereof. For instance, the supplement can be used to treat non-arthritic joint pain, joint discomfort in healthy mammals, lack of joint flexibility in healthy mammals, muscle soreness in healthy mammals, or lack of fitness in healthy mammals. In addition, the supplement of the present disclosure can improve immune health, bone health, or brain health, and may also improve triglyceride and/or cholesterol levels in a healthy mammal and/or a mammal that is regularly undergoing physical activity and/or intense physical activity. Furthermore, the supplement according to the present disclosure may also improve joint health, muscle health and soreness, and cartilage health that is caused by age related decline.

Moreover, the supplement may be suitable for administration to any mammal. For instance, the mammal may be human or canine. The composition can be fed to a mammal of any age such as from parturition through the adult life in the mammal. In various embodiments the mammal may be a human, dog, a cat, a horse, a pig, a sheep, or a cow. In many embodiments, the mammal can be in early to late adulthood. For instance, the active mammal may have an age that is at least 10%, such as least 15%, such as least 20%, such as least 25%, such as least 30%, such as least 35%, such as least 40%, such as least 45%, such as least 50%, such as least 55%, such as least 60%, such as least 65%, such as least 70%, such as least 75%, such as least 85%, such as least 90%, such as least 95% of its expected life span. The mammal may have an age such that it is less than about 95%, such as less than about 90%, such as less than about 85%, such as less than about 80%, such as less than about 75%, such as less than about 70%, such as less than about 65%, such as less than about 60%, such as less than about 55%, such as less than about 50%, such as less than about 45%, such as less than about 40%, such as less than about 35%, such as less than about 30%, such as less than about 25%, such as less than about 20%, such as less than about 15%, such as less than about 10% of its expected life span. A determination of life span may be based on actuarial tables, calculations, or the like.

Furthermore, the present disclosure is also generally directed to a method of improving one or more of endurance, lipid metabolism, and antioxidant status of a mammal. For instance in one aspect, an undenatured type II collagen supplement as discussed herein may be administered to a mammal in an amount sufficient to exhibit an improvement in one or more of endurance, lipid metabolism, and antioxidant status. In one aspect, the mammal may be administered a supplement in an amount of about 1 mg to about 100 mg, such as about 1.5 mg to about 50 mg, such as about 2 mg to about 25 mg, such as about 2.5 mg to about 10 mg, such as about 3 mg to about 5 mg, where the undenatured type II collagen composition is contained in the supplement in the amounts discussed above.

Regardless of the amount of supplement administered, the supplement may be administered to the mammal about 5 times per day or less, such as about 4 times per day or less, such as about 3 times per day or less, such as about 2 times per day or less, such as about once per day, or alternatively, may be administered every other day, every third day, or once or twice per week according to any of the times per day discussed above.

In one aspect, as discussed above, the mammal that is being administered the supplement may also be undergoing physical or intensive physical activity, or may have previously undergone the physical or intensive physical activity. Additionally or alternatively, the mammal may be trained prior to undergoing physical or intensive physical activity as discussed above.

Regardless of the time of administration of the supplement and the level of physical activity, in one aspect, the supplement is administered alone, or in combination with physical or intensive physical activity, such that the mammal exhibits an improvement in one or more of endurance, lipid metabolism, and antioxidant status, as evidenced by the markers discussed above, after both supplementation and physical or intensive physical activity.

Nonetheless, certain embodiments of the present disclosure may be better understood according to the following examples, which are intended to be non-limiting and exemplary in nature.

### Example 1

A total of 21 male Wistar albino rats were randomly divided into 3 groups with seven rats per group. The groups were assigned as: (i) No exercise and no UC-II® brand undenatured type II collagen (Control, standard diet), (ii) Exercise but no UC-II® brand undenatured type II collagen (Exercise, standard diet), and (iii) Exercise+UC-II® brand undenatured type II collagen (4 mg, orally via gavage every day before exercise during the experiment period of 8 weeks).

The exercise protocols were performed on a motor-driven rodent treadmill (MAY-TME, Commat Limited, Ankara, Turkey). The treadmill included a stimulus grid at the back end of the treadmill which provided an electric shock if the animal placed its paw on the grid. The apparatus consisted of a 5-lane animal exerciser utilizing single belt construction with dividing walls suspended over the tread surface. All exercise tests were performed during the same time period of the day to minimize diurnal effects. All rats were pre-trained in order for the animals to be exposed to the treadmill equipment and handling for 1 week based upon the following schedule: (i) 1st day, 10 m/min, 10 min, (ii) 2nd day, 20 m/min, 10 min, (iii) 3rd day, 25 m/min, 10 min, (iv) 4th day, 25 m/min, 20 min and (v) 5th day, 25 m/min, 30 min.

After the 1-week treadmill familiarization to eliminate novel and stress effects, animals in treadmill exercise groups ran on the treadmill 25 m/min, 45 min/day and five days per week for 8 weeks according to the protocol. At the end of the experiment, all rats were subjected to overnight fasting and blood and slow-twitch muscles (soleus and gastrocnemius deep portion) specimens were taken from decapitated animals via cervical dislocation via anesthesia. This procedure was carried out 48 h after the last exercise session to avoid the metabolic effects of the final run. Blood samples were collected by gel biochemical tubes and serum samples were taken and centrifuged at 4° C. at 2370× g for 10 min in a chilled centrifuge. In addition, the tissues obtained from the animals were stored in a deep freeze at −80° C. until analysis.

Distance run average and exhaustion time were measured at the end of each exercise period. Endurance capacity was assayed by treadmill running to fatigue after the end of the training period. In this step, animals were compelled to run on a motorized treadmill, where exhaustion is defined as the inability of the rat to maintain an appropriate pace despite continuous hand prodding for 1 min, at which time the rat was removed from the treadmill (5/lane), and its run time recorded. Results are shown in Table 1 below

The concentration of serum biochemical parameters including, CK, lipid profile and liver and renal functions (urea, creatinine) were measured by ELISA (Elx-800, Bio-Tek Instruments Inc, Vermont, USA). Results are shown in Table 2 below.

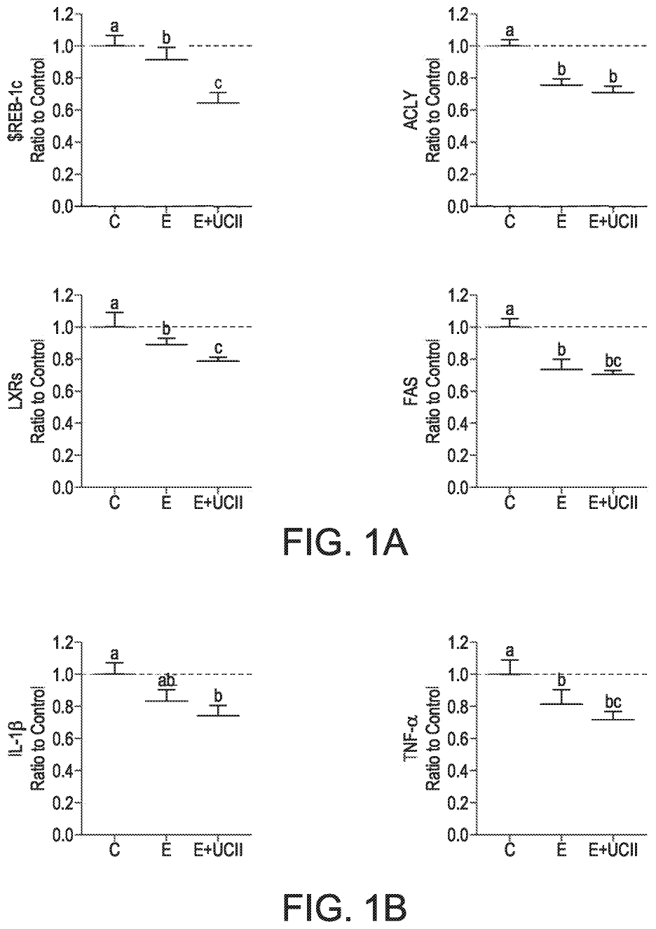

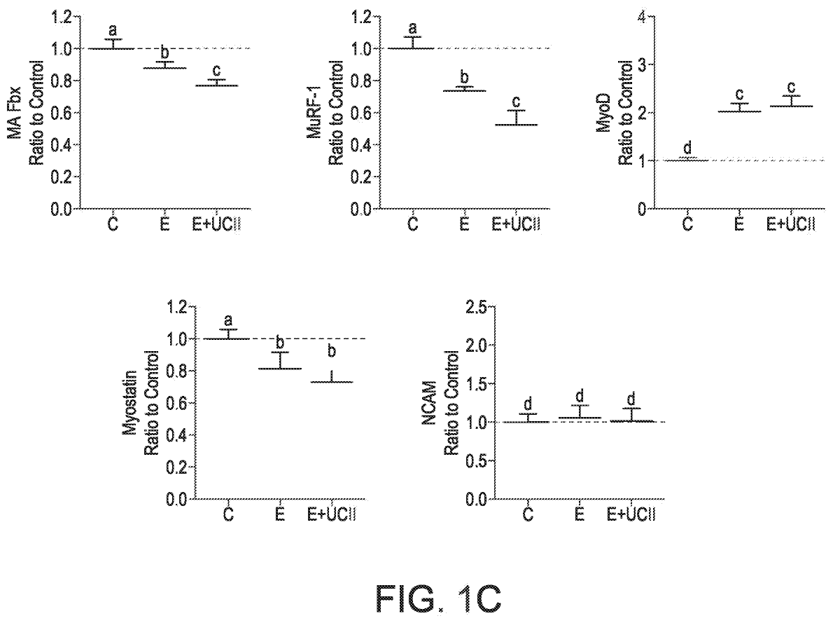

Muscle RING-finger protein-1 (MuRF-1), muscle NCAM, myogenin, myostatin, MyCD, MAFbx, IL-10, TNF-a, SREBP-1c, LXRs, ACLY, FAS levels were determined using the Western blot technique. The densitometric analysis of the relative intensity according to the control group of the western blot bands was performed with β-actin normalization to ensure equal protein loading. Blots were repeated at least three times (n=3) and a representative blot is shown. Data are expressed as a ratio of the control set at 100%. The error bars above the lines point out the standard deviation of the mean. Different symbols indicate statistical differences among the groups (ANOVA and Turkey's post-hoc test; P<0.05). IL-1β, interleukin-1β; TNF-α, tumor necrosis factor α. Results of which are shown in FIGS. 1A-1C

Articular cartilage samples will be analyzed for the expression of COMP, TNF-α, IL-1β, IL-6 will be determined using the Western blot technique. The concentration of serum, lactate and osteocalcin were measured with the rat kit (Cayman Chemical Co., Ann Arbor, Mich., USA) by ELISA (Elx-800, Bio-Tek Instruments Inc, Vermont, USA). Serum myoglobin, COMP, IL-1β, IL-6, TNF-α were determined using by ELISA kit. Results are shown in Table 3 below.

Glucose, total cholesterol, and triglyceride were also measure and recorded for each subject. The results are shown in Table 4 below.

Serum of MDA was analyzed by HPLC. Antioxidant enzymes (SOD, CAT, GSHPx) were measured using the relevant commercial kits according to the enzyme-linked immunosorbent assay (ELISA) method. Results are shown in Table 5 below.

TABLE 1

| | | | Exercise + |
|---|---|---|---|
| | | | Undenatured |
| | | | Type II |
| Details | Control | Exercise | Collagen, 4 mg |
| Initial Body Weight, g | 207.29 ± 4.8 | 217.71 ± 7.6 | 214.00 ± 10.7 |
| Final Body Weight, g | 321.57 ± 4.1 | 283.43 ± 4.5 | 289.86 ± 7.2 |
| Distance Run Average, min | — | 977.29 ± 5.1 | 978.14 ± 8.2 |
| Exhaustion Time, min | 41.29 ± 2.6 | 103.86 ± 4.2 | 123.29 ± 4.0 |

TABLE 2

| | | | Exercise + |
|---|---|---|---|
| | | | Undenatured |
| | | | Type II |
| Details | Control | Exercise | Collagen, 4 mg |
| Total Protein, | 6.56 ± 0.2 | 6.59 ± 0.1 | 6.52 ± 0.2 |
| g/dL | | | |
| Albumin, g/dL | 3.43 ± 0.1 | 3.51 ± 0.1 | 3.53 ± 0.1 |
| Globulin, g/dL | 3.09 ± 0.1 | 3.29 ± 0.1 | 3.19 ± 0.1 |
| Alanine | 97.29 ± 4.6 | 98.43 ± 5.4 | 95.43 ± 3.0 |
| Transaminase | | | |
| (ALT), U/L | | | |
| Aspartate | 118.43 ± 6.2 | 116.86 ± 6.3 | 114.43 ± 7.8 |
| transaminase | | | |
| (AST), U/L | | | |
| Total Bilirubin, | 0.24 ± 0.01 | 0.24 ± 0.01 | 0.24 ± 0.01 |
| mg/dL | | | |
| Creatinine kinase | 125.80 ± 1.8 | 193.60 ± 2.9 | 179.98 ± 2.8 |
| (CK), IU/L | | | |
| Creatinine, mg/dL | 0.48 ± 0.03 | 0.47 ± 0.02 | 0.48 ± 0.04 |
| Blood Urea | 20.47 ± 0.8 | 20.86 ± 0.2 | 19.44 ± 0.6 |
| Nitrogen (BUN), | | | |
| mg/dL | | | |

TABLE 3

| | | | Exercise + |
|---|---|---|---|
| | | | Undenatured |
| | | | Type II |
| Details | Control | Exercise | Collagen, 4 mg |
| Lactate, mmol/L | 2.80 ± 0.1 | 5.76 ± 0.1 | 4.96 ± 0.2 |
| Osteocalcin, ng/mL | 26.93 ± 0.5 | 30.75 ± 0.5 | 31.07 ± 0.7 |
| Myoglobin, ng/mL | 21.95 ± 0.6 | 42.34 ± 0.9 | 36.59 ± 0.6 |
| Cartilage Oligomeric | 6.86 ± 0.2 | 6.98 ± 0.3 | 6.48 ± 0.3 |
| Matrix Protein (COMP), | | | |
| ng/mL | | | |
| IL-1β, pg/mL | 19.63 ± 0.5 | 19.89 ± 0.6 | 18.97 ± 0.7 |
| IL-6, pg/mL | 10.05 ± 0.3 | 9.06 ± 0.4 | 8.56 ± 0.5 |
| Tumor Necrosis Factor | 19.72 ± 0.4 | 18.42 ± 0.7 | 17.32 ± 0.5 |
| alpha (TNFα), | | | |
| pg/mL | | | |

TABLE 4

| | | | Exercise + |
|---|---|---|---|
| | | | Undenatured |
| | | | Type II |
| Details | Control | Exercise | Collagen, 4 mg |
| Glucose, | 108.29 ± 3.2 | 102.14 ± 1.8 | 102.43 ± 1.6 |
| mg/dL | | | |
| Total | 98.51 ± 1.7 | 92.06 ± 1.6 | 95.70 ± 1.5 |
| Cholesterol, | | | |
| mg/dL | | | |
| Triglyceride, | 103.51 ± 1.2 | 96.58 ± 1.7 | 102.40 ± 1.8 |
| mg/dL | | | |

TABLE 5

| | | | Exercise + |
|---|---|---|---|
| | | | Undenatured |
| | | | Type II |
| Details | Control | Exercise | Collagen, 4 mg |
| Serum | 0.76 ± 0.02 | 0.68 ± 0.01 | 0.65 ± 0.02 |
| Malondialdehyde | | | |
| (MDA) μmol/L | | | |
| Muscle MDA, | 1.91 ± 0.05 | 1.67 ± 0.04 | 1.63 ± 0.05 |
| nmol/mg | | | |
| Superoxide | 70.88 ± 1.6 | 82.28 ± 1.8 | 73.91 ± 2.1 |
| Dismutase | | | |
| (SOD), U/mL | | | |
| Catalase (CAT), | 140.82 ± 2.8 | 153.00 ± 2.8 | 141.52 ± 2.2 |
| U/mL | | | |
| Glutathione | 71.13 ± 2 | 82.00 ± 2.6 | 74.16 ± 2.1 |
| peroxidase | | | |
| (GSHPx), U/ml | | | |

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A supplement for improving one or more of endurance, lipid metabolism, and antioxidant status in a mammal, the supplement comprising:
a type II collagen composition, the type II collagen composition comprising undenatured collagen, hydrolyzed collagen, or a combination thereof;
wherein the type II collagen composition is present in the supplement in an amount sufficient to:
increase endurance of a mammal administered the supplement by about 5% or more as compared to a mammal that has not been administered the supplement,
improve lipid metabolism of a mammal administered the supplement by about 5% or more as compared to a mammal that has not been administered the supplement, and/or
decrease oxidative stress of a mammal administered the supplement by about 5% or more as compared to a mammal that has not been administered the supplement.

2. The supplement of claim 1, wherein the type II collagen composition is present in the supplement in an amount sufficient to increase a run time to exhaustion in a mammal administered the supplement by about 5% or more as compared to a mammal that has not been administered the supplement.

3. The supplement of claim 1, wherein the type II collagen composition is present in the supplement in an amount sufficient to decrease sterol regulatory element binding protein-1c (SREBP-1c) in a mammal administered the supplement by about 5% or more as compared to a mammal that has not been administered the supplement.

4. The supplement of claim 1, wherein the type II collagen composition is present in the supplement in amount sufficient to decrease malondialdehyde levels in a mammal administered the supplement by about 5% or more as compared to a mammal that has not been administered the supplement.

5. The supplement of claim 1, wherein the type II collagen composition further comprises one or more different types of collagen in addition to the type II collagen.

6. The supplement of claim 5, wherein the one or more different types of collagen include native type II collagen, collagen peptides, or a mixture thereof.

7. The supplement of claim 1, wherein the type II collagen is undenatured type II collagen, and is derived from chicken sternum cartilage.

8. The supplement of claim 1, wherein the undenatured type II collagen comprises about 1% to about 95% of the type II collagen composition.

9. The supplement of claim 1, wherein the undenatured type II collagen has a total oxygen radical absorbance capacity of about 200 μmol TE/g or greater, as measured according to ORAC 6.0.

10. The supplement of claim 1, wherein the supplement is in the form of, or is incorporated into, a food or beverage.

11. A method of improving one or more of endurance, lipid metabolism, and antioxidant status in a mammal, comprising:
administering a supplement to the mammal, the supplement comprising a type II collagen composition, the type II collagen composition comprising undenatured collagen, hydrolyzed collagen, or a combination thereof,
wherein the type II collagen composition is present in the supplement in an amount sufficient to:
increase endurance of a mammal administered the supplement by about 5% or more as compared to a mammal that has not been administered the supplement,
improve lipid metabolism of a mammal administered the supplement by about 5% or more as compared to a mammal that has not been administered the supplement, and/or
decrease oxidative stress of a mammal administered the supplement by about 5% or more as compared to a mammal that has not been administered the supplement.

12. The method of claim 11, wherein both the mammal administered the supplement, and a mammal that has not been administered the supplement, have previously undergone physical activity, are currently undergoing physical activity, or have previously undergone physical activity and are currently undergoing physical activity.

13. The method of claim 11, wherein the method further includes subjecting both the mammal administered the supplement, and a mammal that has not been administered the supplement to a time of physical activity, wherein the increase in endurance, the improved lipid metabolism, and/or the decrease in oxidative stress is measured after the time of physical activity.

14. The method of claim 13, wherein the time of physical activity is at least one week, and wherein the increase in endurance, the improved lipid metabolism, and/or the decrease in oxidative stress is measured at the end of the at least one week.

15. The method of claim 11, wherein the type II collagen composition is present in the supplement in an amount sufficient to increase a run time to exhaustion in a mammal administered the supplement by about 5% or more as compared to a mammal that has not been administered the supplement.

16. The method of claim 11, wherein the type II collagen composition is present in the supplement in an amount sufficient to decrease sterol regulatory element binding protein-1c (SREBP-1c) in a mammal administered the supplement by about 5% or more as compared to a mammal that has not been administered the supplement.

17. The method of claim 11, wherein the type II collagen composition is present in the supplement in amount sufficient to decrease malondialdehyde levels in a mammal administered the supplement by about 5% or more as compared to a mammal that has not been administered the supplement.

18. The method of claim 11, wherein the type II collagen composition is administered to the mammal daily.

19. The method of claim 11, wherein the type II collagen composition is administered to a mammal while the mammal is undergoing physical activity.

20. The method of claim 11, wherein the type II collagen composition is administered to the mammal as a food or beverage.
